(19) United States
(12) Patent Application Publication
(10) Pub. No.: US 2015/0018781 A1
(43) Pub. Date: Jan. 15, 2015

(54) SYSTEMS, DEVICES, AND METHODS FOR TOPICAL DRUG DELIVERY TO THE EYE

(71) Applicants: CALIFORNIA INSTITUTE OF TECHNOLOGY, Pasadena, CA (US); THE REGENTS OF THE UNIVERSITY OF CALIFORNIA, Oakland, CA (US)

(72) Inventors: Derek Rinderknect, Arcadia, CA (US); Michael J. Geske, San Francisco, CA (US); Morteza Gharib, Altadena, CA (US); Daniel M. Schwartz, San Francisco, CA (US)

(73) Assignees: CALIFORNIA INSTITUTE OF TECHNOLOGY, Pasadena, CA (US); THE REGENTS OF THE UNIVERSITY OF CALIFORNIA, Oakland, CA (US)

(21) Appl. No.: 14/249,309

(22) Filed: Apr. 9, 2014

Related U.S. Application Data
(60) Provisional application No. 61/810,614, filed on Apr. 10, 2013.

(51) Int. Cl. A61F 9/00

(52) U.S. Cl. CPC A61F 9/0026
USPC 604/298

(57) ABSTRACT

Systems, devices, and methods are provided for delivering a therapeutic substance to the eye of a subject with an automated apparatus. The apparatus can resemble a pair of eyeglasses that can assist in the alignment of a substance ejecting nozzle and energy sensor with the eye. The apparatus can eject the therapeutic substance and immediate response to the detection of a blink of the user's eye. The therapeutic substance can be ejected in a number of different forms including as a single droplet containing the entire prescribed dosage.

### CROSS-REFERENCE TO RELATED APPLICATIONS

This application claims the benefit of and priority to U.S. Provisional Patent Application Ser. No. 61/810,614, filed on Apr. 10, 2013, which is incorporated by reference herein in its entirety for all purposes.

### FIELD

The subject matter described herein relates generally to systems, devices, and methods for the topical delivery of drugs and other substances to the eye.

### BACKGROUND

A number of prescription drugs are available for topical application to the eye to treat a variety of diseases. The application of these drugs in a reliable, controllable, and measurable fashion, however, presents many challenges. The drugs are commonly applied by way of an eye dropper, where the force of gravity carries the drug into the eye. Patients using an eye dropper frequently apply the dosage away from the center of the eye or miss the eye altogether, resulting in less than the full dosage being delivered. This problem is exacerbated if the patient is elderly or in poor health and lacks the requisite coordination or steadiness. The patient may also hold his or her eye open for an extended period of time while lining up the dropper for application, thereby drying the eye out and resulting in excessive blinking and/or tears that dilute and transfer the dosage out of the eye.

These challenges can lead to uncertainty as to whether the prescribed dosage was actually delivered at the prescribed intervals, which can significantly hinder an assessment of the effectiveness of the drug and can even lead to detrimental changes in a patient's treatment program. If a significant volume of the drug is not actually absorbed into the body, not only does this hinder the patient's treatment but it also results in significant over-expenditure, as many of the drugs are often expensive.

A number of devices have been proposed to address the challenges presented by use of the standard eye dropper, such as that described in US Patent Application Publication 2012/0143152 to Hunter et al. These devices, however, tend to be cumbersome, ineffective, excessively complex, difficult to align, and/or overly expensive. Some of these proposals come in the form the handheld devices that, even with automated alignment techniques, can be difficult to first align and then keep aligned to the eye, especially given that the patient's that have the most difficulty in topical drug application are the elderly and the sick. These and other proposals transfer the drug to the eye in the form of a sprays, mists, or fogs that, by their nature, tend to lack precision and have small particulate sizes. A significant amount of the drug can fail to be absorbed by the body, either by landing away from the center of the eye (or even off of the eye) as a result of imprecise delivery or by being carried off (e.g., floating away) as a result of the susceptibility of the substantial number of small particulates to minor air currents. Still other proposals fail to provide adequate safeguards against the drying out of the eye before drug application and/or against excessive blinking of the eye just after application.

As a result, needs exists for improved systems, devices, and/or methods for the topical delivery of drugs and other therapeutic substances to the eye.

### SUMMARY

Many example embodiments of systems, devices, and methods for the topical delivery of drugs and other substances to the eye are disclosed herein. Some of these embodiments utilize a drug delivery apparatus that includes a wearable frame, an ejection device, a sensor, and control circuitry. The wearable frame can resemble an eyeglass-frame and can provide a simple “hands-free” manner of alignment of the ejection device to the subject's eye.

These and other embodiments can utilize the emission and detection of infrared light, in some cases pulsed infrared light, to detect whether the subject's eye is open or closed. The drug delivery apparatus can actuate the ejection of the drug toward the eye upon detection of a blink by the patient, which increases the likelihood that the eye will be open when the drug makes contact with the subject, and also guards against drying-out of the eye before application.

Certain embodiments of the drug delivery apparatus include a single aperture in the ejection device and are configured to apply the entire dosage in one single drop, or a small number of single drops, propagated from the ejection device. The drop size can be adjusted to meet the prescribed dosage. This larger drop approach overcomes many of the disadvantages associated with sprays, mists, and fogs.

Still other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments in this or any section hereof be construed as limiting the appended claims, absent express recitation of those features therein.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a perspective view of an example embodiment of a delivery apparatus for administering a therapeutic substance to a subject.

FIG. 2A is a cross-sectional view of an example embodiment of an ejection device.

FIG. 2B is a photograph of a portion of another example embodiment of the delivery apparatus from a subject's perspective.

FIGS. 2C-E are cross-sectional views of an example embodiment of a pump.

FIGS. 2F-H are cross-sectional views of an example embodiment of a pump-less ejection device.

FIG. 3 is a block diagram of another example embodiment of the delivery apparatus in relation to a subject's eye.

FIG. 4A is a block diagram of an example embodiment of signal conditioning circuitry.

FIG. 4B is a schematic diagram of another example embodiment of signal conditioning circuitry.

FIGS. 5A-D are time lapse photographs of the ejection of a single drop of therapeutic substance from a nozzle of another example embodiment of the delivery apparatus.

FIG. 6A is a graph of example responses of an example embodiment of a piezoelectrically actuatable membrane to actuation signals having different magnitudes.

FIG. 6B is a graph of example responses of an example embodiment of a piezoelectrically actuatable membrane to actuation signals having different durations.

### DETAILED DESCRIPTION

The present subject matter is not limited to the particular embodiments described, as those are only examples and may, of course, vary. Likewise, the terminology used herein is for the purpose of describing particular embodiments only, and is not intended to be limiting, since the scope of the present disclosure will be limited only by the appended claims.

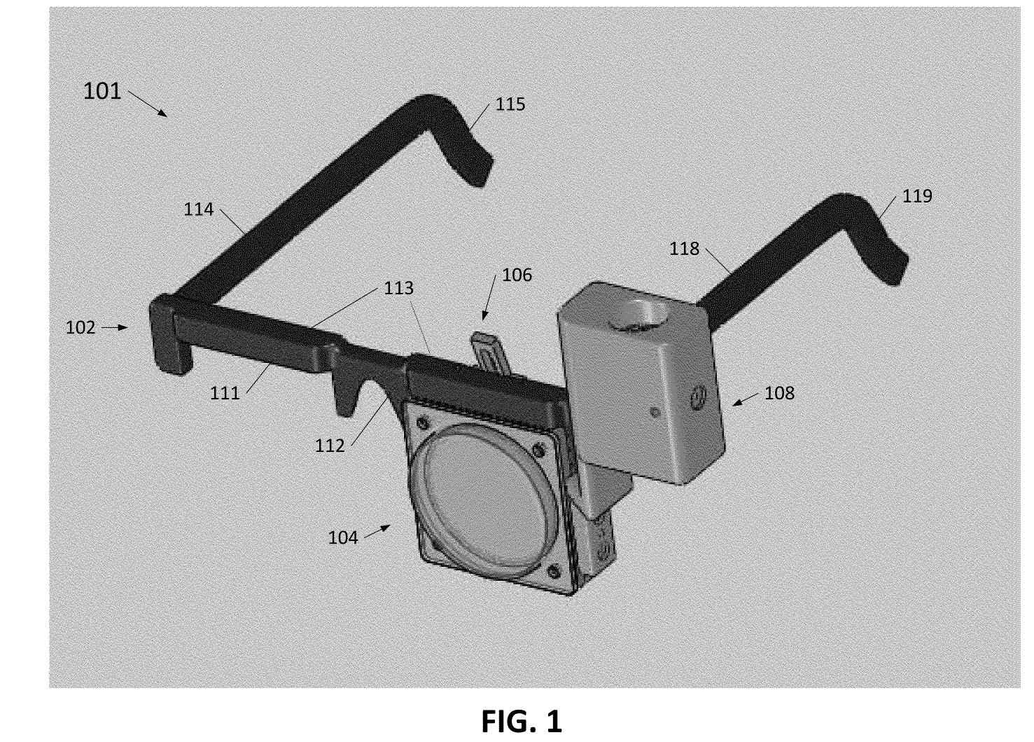

FIG. 1 is a perspective view depicting an example embodiment of a delivery apparatus 101 that can be used within a more encompassing delivery system 100. In this embodiment, delivery apparatus 101 includes a wearable frame 102 with an ejection device 104, a sensor unit 106, and an adapter unit 108. Apparatus 101 is capable of delivering a therapeutic substance to the subject's eye in a controllable, adjustable, and verifiable fashion. The term “therapeutic substance” should be construed broadly to encompass all substances that may be used in a subject's treatment therapy, including but not limited to man-made and naturally occurring drugs.

Delivery apparatus 101 can be used to treat a host of tissue conditions, including but not limited to glaucoma, infection, dry eye disease, corneal abrasions, intraocular inflammation (uveitis), postoperative prophylaxis against infection and inflammation, and others. Apparatus 101 can be used by the subject directly in any environment (e.g., home, business, etc.) and can also be used by a medical professional (e.g., practitioner or technician) in a clinical setting, such as for the administration of therapeutic substances for the dilation of the pupil or for anesthesia of the ocular surface to aid in examination of or procedures on the subject patient.

Ejection device 104, sensor unit 106, and adapter unit 108 are shown in a location corresponding to the subjects left eye, but these components can also be positioned on the opposite side of frame 102 for treatment of the subject's right eye, as an alternative to or in addition to the components shown. In some embodiments, a single adapter unit 108 can be used for eyepieces 104 located on both the left and right sides.

Wearable frame 102 can take many forms. In FIG. 1, wearable frame 102 is in the form of eyeglasses or is configured an eyeglass-type fashion, but of course does not require the presence of eyeglass lenses. Frame 102 includes a first, center strut 111 that is configured for placement across the subject's face and has a centrally-located nose rest feature 112, which can have the shape of an inverted U or V. Center strut 111 includes base portions 113 that are positioned on either side of nose rest 112 and can serve as a mounting for eyepiece 104. A second strut 114 and a third strut 118 are pivotably coupled (e.g., with a mechanical or living hinge) or fixed to opposite ends of center strut 111 and are configured for placement along opposite sides of the subject's head. Struts 114 and 118 can include rear hook-like portions 115 and 119, respectively, for engaging with the subject's ears.

In other embodiments, apparatus 101 can be configured to extend over only one eye, for example, such as by one of the side struts 114 or 118 and the adjacent base portion 113, but leaving nose rest 112 and the base portion 113, and side strut on the side of the head with the eye to be treated. Also, other manners of attachment of apparatus 101 can be used instead of side struts 114 and 118, including one or more straps, bands, clamps, and so forth. The embodiments described herein can also be used in other devices that do not include a wearable frame.

Frame 102 can be fabricated from similar materials and in a similar manner to prescription or sunglass frames. In certain embodiments, these frames are custom tailored to the subject's face ensuring that both sensor unit 106 and ejection device 108 are properly aligned. For example, frame 102 can be 3D printed based on the facial geometry of the subject. In certain embodiments, after placing frame 102 on the subject's face, either or both of sensor unit 106 and ejection device 108 can be adjustably aligned to the subject's eye and then locked in place.

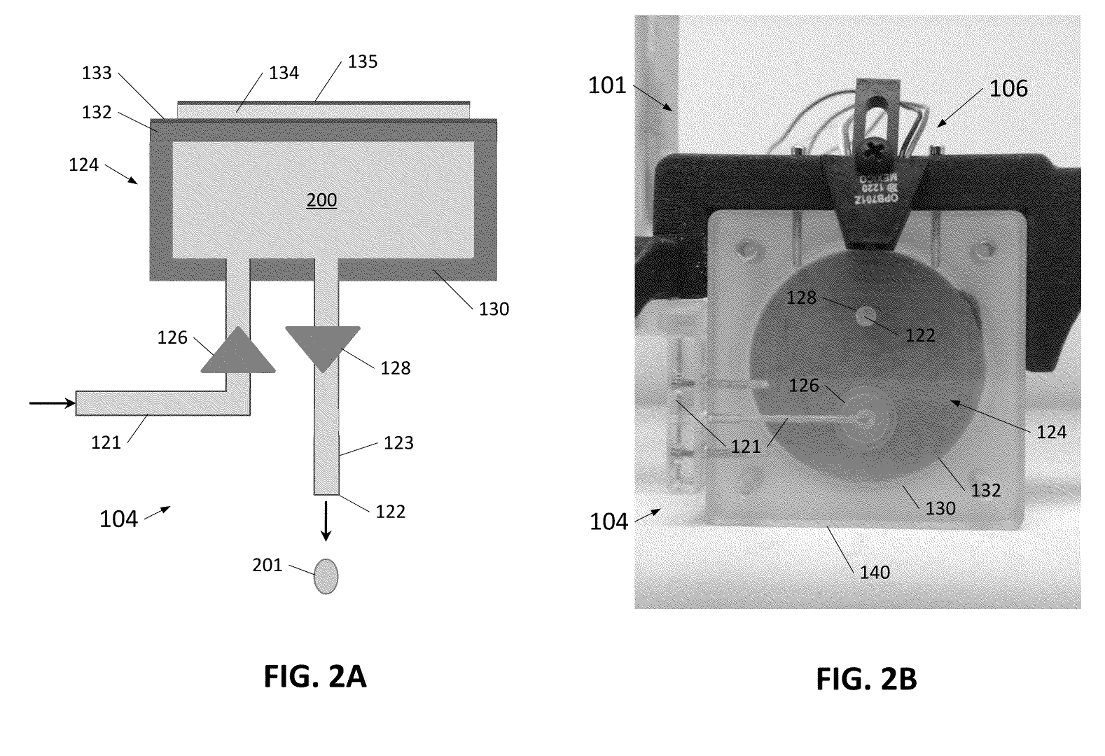

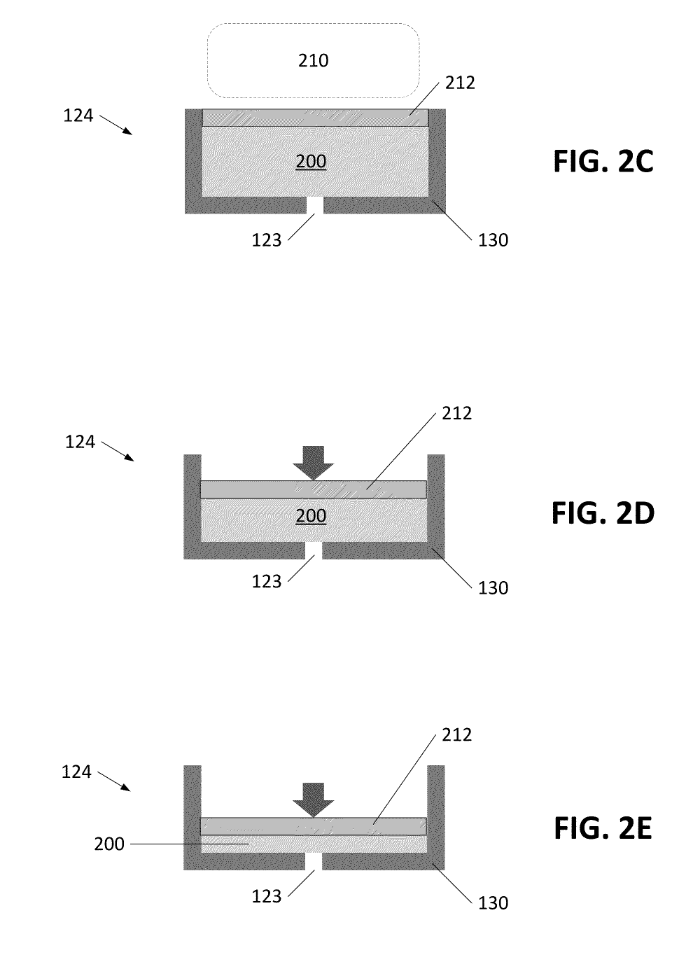

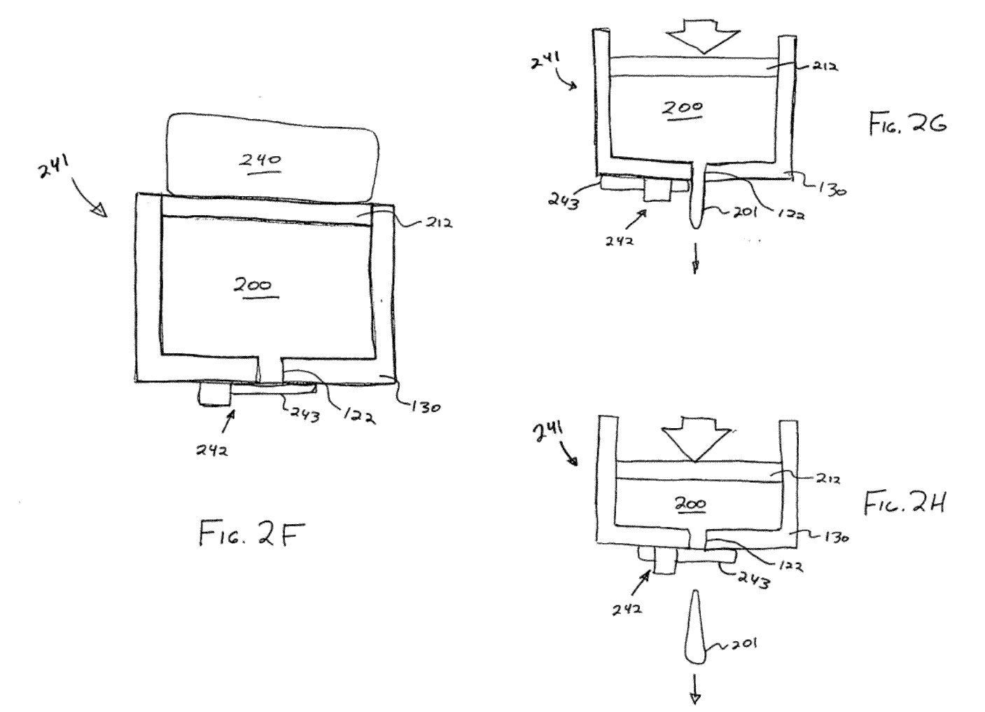

Ejection device 104 includes a pump 124 that receives the therapeutic substance over a fluidic channel or pathway from adapter unit 108 and causes it to be ejected through nozzle 122 to the eye. FIG. 2A is a cross-sectional view of an example embodiment of an ejection device 104 having a piezoelectrically actuatable pump 124. In other embodiments, pump 124 can operate piezoelectrically, electromagnetically, electrostatically, pneumatically, hydraulically, or can be driven by a solenoid.

Pump 124 includes a piezo-electrically actuatable device 134 (e.g., a crystal, ceramic, polymer, and the like) in a planar (or sheet-like) configuration with a circular profile. Other configurations and shapes can also be used depending on the needs of the application. A control (or actuation) signal is provided to device 134 by way of electrodes 133 and 135 in position on opposite sides of device 134. Here, electrodes 133 and 135 have similar profiles to device 134 and can flex or displace with device 134. Biasing of electrodes 133 and 135 with the control signal induces movement of device 134, which in turn causes corresponding movement in flexible wall 132, which is located at the rear of pump chamber 130.

Flexible wall 132 can be fabricated from the same material as the walls of chamber 130, or from a different material. Flexible wall 132 can be integral to chamber 130, such that chamber 130 and wall 132 are of monolithic construction, or wall 132 can be a separate piece securely coupled with chamber 130, such as with adhesive, coupling elements (screws, rivets, clamps, etc.) and the like. Flexible wall 132 can be in the form of a membrane, sheet, plate, or the like.

A rigid displaceable wall can also be used, for instance, by acting as a piston or other rigid driving mechanism that travels back and forth to adjust the volume of chamber 130. An example embodiment of such a configuration is depicted in the cross-sectional views of FIGS. 2C-E. A rigid (or semi-rigid) wall or piston 212 is located at the rear of chamber 130. Rigid wall 212 can be driven by an actuator 210, such as, for instance, a solenoid or voice-coil. FIGS. 2C-E depict progressive movement of wall 212 in a downward motion from the force of actuator 210, which progressively empties substance 200 from chamber 130. The greater the downward displacement of wall 212 (or 132 in the previous embodiment), the greater the dose ejected from chamber 130. These embodiments having walls 132 and 212 can be used with any of the pump embodiments described herein.

Pump chamber 130 can of rigid or semi-rigid construction, as is readily understood by those of ordinary skill in the art. Chamber 130 can have be compressed and expanded by the movement of rear wall 132 or 212, or by the movement of one or more of the front, top, bottom, rear, and/or side walls, and any combination thereof. In certain embodiments, chamber is of rounded construction (such as a flexible tube or ball-like cavity).

Also shown in FIG. 2A is inlet 121 through which the therapeutic substance 200 can travel from an internal reservoir or attached container (not shown), through a one-way valve 126, and into pump chamber. An outlet 123 extends from chamber 130 through a second one-way valve 128. The therapeutic substance can be discharged from chamber 130 through outlet 123 such that the desired volume is ejected from nozzle 122.

Movement of device 134 (and wall 132) away from nozzle 122 increases the volume of chamber 130 and causes therapeutic substance 200 to flow into chamber 130 through valve 126. The other one-way valve 128 prevents therapeutic substance 200 (or any priming fluid) from moving from nozzle 122 back into chamber 130. Movement of device 134 (and wall 132) towards nozzle 122 decreases the volume of chamber 130 and causes therapeutic substance 200 to flow from chamber 130 through outlet 123 and out of nozzle 122. This is shown in FIG. 2A by the dosage exiting in the form of a single drop 201.

FIG. 2B is a photograph of a partially disassembled apparatus 101 taken from the subject's perspective to show ejection device 104 and sensor unit 106 in greater detail. Pump 124 is located in a housing 140, which in this embodiment is formed by two polycarbonate bodies (configured as plates) that are coupled together in a sealed fashion. Other configurations and materials (polymers, stainless steel, ceramics, etc.) can be used for housing 140 as well. Inlet 121 leads through one of the polycarbonate plates and allows a drug to be carried from an internal reservoir (not shown) that is in turn connected to the drug container (also not shown) to a one-way valve 126, which in turn leads immediately to pump chamber 130, which is present between the opposing plates and has a circular periphery in the same general area as (if not slightly larger than) the circular gray object, which is electrode 133. One way valve 128 is shown directly beneath nozzle 122.

Nozzle 122 is in a position on ejection device 104 that corresponds to the center of the subject's eye when frame 102 is being worn. As noted herein, other parts of the eye (or around the eye) can be targeted as well. Nozzle 122 is oriented to eject the therapeutic substance at an angle perpendicular to the surface of FIG. 2B (i.e., directly out of the sheet). Nozzle 122 can include any number of one or more apertures through which the therapeutic substance can be ejected, and that substance can be ejected in any desired form, including as a single drop (as would be the case with a single aperture as shown here), as a multi-particulate spray that can propagate with a substantially constant cross-sectional diameter or with an expanding cross-sectional diameter such as would be the case with a flaring or conical pattern, or as a generally shapeless mist or fog.

Nozzle 122 can be integrated with ejection device 104 (in a monolithic or pseudo-monolithic fashion) or can be a discrete component that is interchangeable with other nozzles 122 of varying dimension or type, which can allow apparatus 101 to convert from single drop ejection to spray ejection and so forth.

Nozzle 122 may be fabricated from any of the following biocompatible polymers: polysulfone, silicone, ultra-high-density polyethylene (UHDPE), polytetrafluoroethylene (PTFE), polycarbonate, polyether ether ketone (PEEK), cyclic olefin copolymer (COC), and the like. The nozzle's one or more apertures may be fabricated from the same material as nozzle 122 or a different material, such as a metal like stainless steel or 6Al4V titanium, or with precious stones such as ruby, diamond, or sapphire. Although not limited to such, the one or more nozzle apertures can each have a diameter in the range of 1-500 microns (μm). In certain embodiments the nozzle has a single aperture with a diameter in the range of 50-400 μm, 101-400 μm, 150-350 μm, or 200-300 μm, to name a few. Outlet 123 can have a taper with a decreasing diameter as progressing towards nozzle 122, can taper with an increasing diameter as progressing towards nozzle 122, or can have a constant cross-sectional diameter as depicted in FIG. 2A.

The peripheral profile of the nozzle aperture(s) can be rounded, such as circular or elliptical and the like. In a preferred embodiment, nozzle 122 has an aperture with a single circular profile (from the perspective of FIG. 2B) with squared edges (edges as shown in FIG. 2A), where outlet 123 also has a constant diameter along its length (with an exception possible for valve 128). As an alternative to squared aperture edges, flared or tapered edges can also be used. Although not limited to such, nozzle 122 can have a length of 0.01-15000 μm. In certain embodiments, nozzle 122 has a length between 1-500 μm. The surface chemistry of nozzle 122 can be modified or coated as desired to enhance substance detachment and surface tension characteristics.

Apparatus 101 can include one or more one-way valves 126 or 128 that assist in the movement of the therapeutic substance through the internal fluidic channels of the device. These one-way valves 126 and 128 may be of any desired type, including but not limited to umbrella, duck-bill, ball valve, multi-leaflet, flap valves, or Belleville valves. One-way valves 126 and 128 are oriented to ensure the fluid is continually moving out of apparatus 101.

For example, when flexible wall 132 is actuated the pressure generated in the adjacent fluidic chamber 130 causes one-way valve 126 on the inlet to close and one-way valve 128 on the outlet to open resulting in a surge of fluid towards nozzle 122. When the membrane is actuated in the reverse direction, one-way valve 128 on the outlet closes and one-way valve 126 on the inlet opens allowing new fluid to be drawn into the fluidic chamber. In a similar embodiment multiple one-way valves 126 and 128 are located on both the inlet and outlet sides of pump 124. Although not shown, ejection device 104 can have one or more on/off valves allowing the isolation of the therapeutic substance therein. The on/off valve can include a closure such as an interference fitting or screw on cap. The on-off valves can be used in conjunction with one or more one-way valves 126 and 128.

Ejection device 104 can also operate in a pump-less manner. FIGS. 2F-H are cross-sectional views depicting an example embodiment of a release device 241 having a chamber 130 and rigid (or semi-rigid) displaceable wall 212 like the embodiment of FIGS. 2C-E. A pressure generating device 240 is located behind wall 212 and applies a force to wall 212 to put substance 200 under pressure within chamber 130. FIG. 2F depicts an electrically actuatable release valve 242 (e.g., an on-off valve) having a movable gate 243 in a closed position covering nozzle 122. With contents 200 under pressure, gate 243 can be slid or moved across nozzle 122 in a rapid fashion to allow pressurized contents 200 to exit nozzle 122, as depicted in FIG. 2G. Valve 242 is then rapidly closed by moving gate 243 back to the original position as depicted in FIG. 2H. The duration of which valve 242 is open, and the rate of release of substance 200 (determined by the applied pressure, dimensions of nozzle 122, etc.), can be controlled to release only the desired amount (dose) of substance 200.

Referring back to FIG. 1, sensor unit 106 is used to detect whether the subject's eye is in an open state or closed state, i.e., whether the eyelid is up or down. In many embodiments, sensor unit 106 is used to detect a condition indicative of the occurrence of a blink of the subject's eye. This can entail detection of movement of the eyelid from an up to a down position, from a down to an up position, movement from an up to a down position and back again, or movement from an up to a down position and back again within a predetermined (e.g., rapid) time window.

Sensor unit 106 can include a sensor for sensing photonic energy that can be used to determine the state of the eye. This photonic energy can include ultraviolet light, optical light, infrared (IR) light, or photons at any other frequency. Sensor unit 106 can also detect other forms of non-photonic energy as desired. The energy can be sourced from the environment, e.g., sunlight or indoor lighting, or can be sourced from an energy emitter that is included as part of sensor unit 106. Although not limited to such, in certain embodiments the wavelength of energy emitted is 300 nanometers to 2 microns.

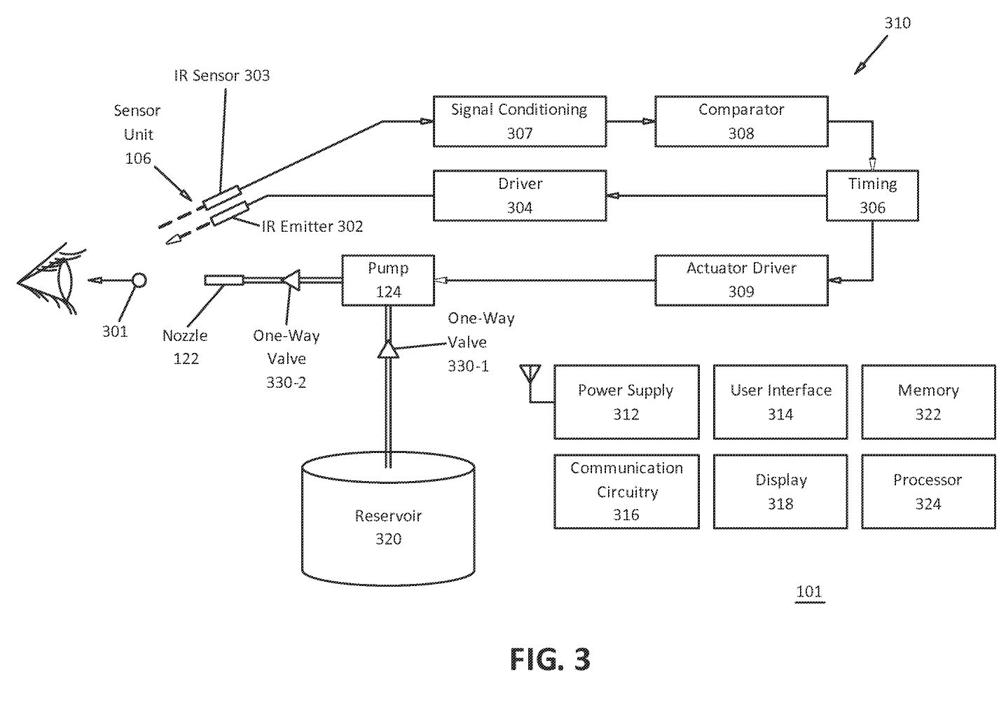

Sensor unit 106 is shown in FIG. 3, which is a block diagram depicting apparatus 101. Here, sensor unit 106 includes IR emitter 302, which is the source of IR energy detected by IR sensor 303. A timing circuit (or software) 306 activates driver 304, which in turn causes IR emitter 302 to emit IR energy towards the subject's eye. IR emitter 302, which may, for example, be a light emitting diode (LED), can be operated in a continuous or intermittent fashion. For example, IR emitter 302 can be operated in a pulsed fashion in order to allow apparatus 101 to distinguish the emitted energy from ambient sources and random noise. The frequency of pulse emission can be greater than 500 hertz, or in the range of 500 hertz to one megahertz, or in some embodiments in the range of 2 kilohertz to 60 kilohertz, although any other frequency that permits distinguishing the emitted IR energy from the surrounding environmental sources can be used. The pulsing of emissions also allows apparatus to conserve energy, e.g., battery power. The IR energy pulses can have a duty cycle in the range of one to 50% (on), and in some embodiments in the range of 5 to 15% (on), although other duty cycles can be used.

IR sensor 303 can be any device capable of sensing IR energy, such as one or more photodiodes (avalanche or otherwise), phototransistors, photodarlingtons, charge coupled devices (CCD), CMOS imagers, or any combination thereof. If multiple devices are used they can be placed in an arrayed fashion. If desired, the peak spectral wavelengths of the emitter can match that of the sensor.

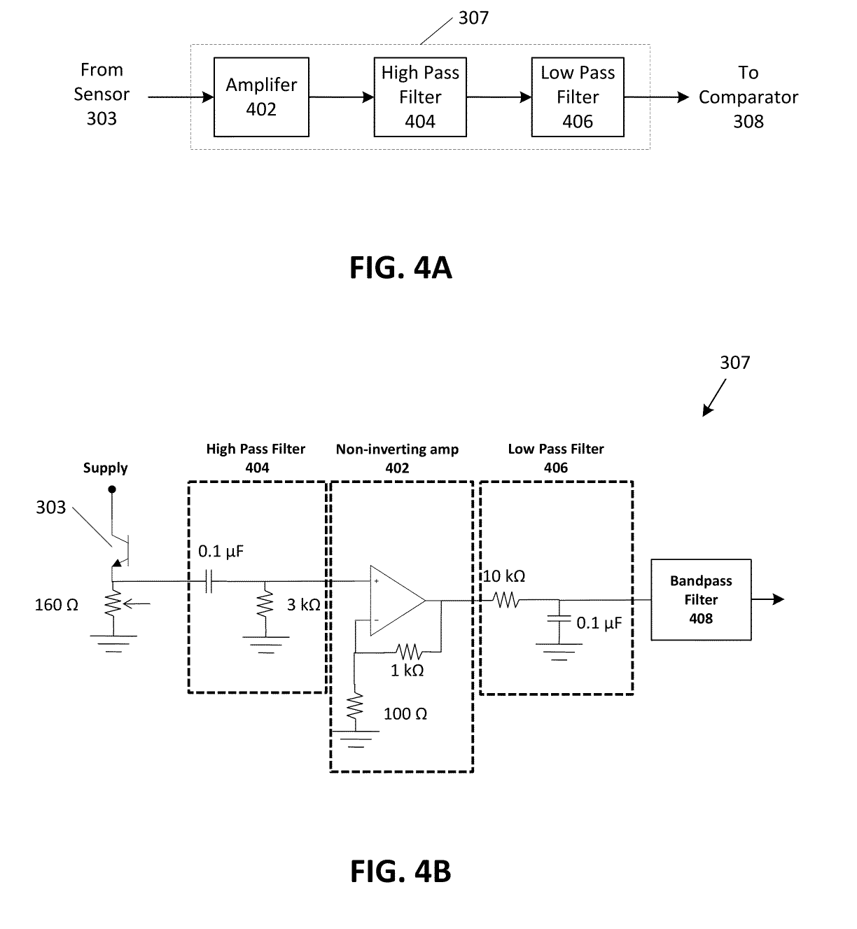

As can be seen in FIG. 3, apparatus 101 includes a variety of electrical or control circuitry 310. Sensor 303 outputs a signal to signal conditioning circuitry 307, which can include one or more amplifiers and one or more filters for conditioning the signal for further processing. FIG. 4A is a block diagram depicting an example embodiment of signal conditioning circuitry 307 configured to isolate the IR energy that was emitted from emitter 302 at a known pulse frequency, reflected from the subject's eye, and sensed by sensor 303. In this embodiment, signal conditioning circuitry 307 includes an amplifier 402 for amplifying the output of sensor 303, high pass filter 404 for removing low-frequency and DC components beneath the known pulse frequency, and low pass filter 406 for removing any high frequency components above the known pulse frequency. Alternate arrangements of amplifiers and filters can also be used.

One such alternate arrangement is depicted in FIG. 4B, which is a schematic view of sensor 303 and signal conditioning circuitry 307. Here, sensor 303 is a photodarlington that outputs to high pass filter 404, followed by non-inverting amplifier 402, low pass filter 406, and finally bandpass filter 408, which may be a Butterworth filter or the like. The component values given here are purely for example and those of ordinary skill in the art will readily realize that other values can be used depending on the application.

Referring back to FIG. 3, the output from signal conditioning circuitry 307 is passed to comparator 308. Comparator 308 compares the received signal to one or more conditions (discussed below) that are indicative of a state of the eye. As mentioned, an example of this can be the sensing of a condition considered to be evidence that a blink is likely occurring, which will typically produce a rapid and short-lasting change (e.g., a spike) in the amount of IR energy sensed by sensor 303. Blink determination based the use of IR energy is described in greater detail in Ryan et al., “A long-range, wide field-of-view infrared eyeblink detector,” Journal of Neuroscience Methods 152 (2006) 74-82, which is incorporated by reference herein in its entirety for all purposes. The function of comparator 308 can be performed in analog or digital domains, by discrete or dedicated circuitry or by software executed by one or more processors (if in digital format).

The conditions used by comparator 308 can be adjustable to accommodate variations between subjects, such as variations in skin color and so forth. In some embodiments, a routine is run by apparatus 101 that automatically calibrates comparator 308 to the reflectivity of the subject's skin. Any desired condition can be used to initiate ejection. For example, the conditions can include, but are not limited to: the sensed energy increasing by a threshold percentage or amount; the sensed energy decreasing by a threshold percentage or amount; the sensed energy increasing to a qualifying maximum and then decreasing; the sensed energy increasing to a qualifying maximum and then decreasing to a threshold percentage of the maximum (e.g., 90%, 85%, or 80% and so forth) or by a threshold amount from the maximum; a threshold rate of change in the sensed energy; a threshold rate of rate of change in the sensed energy; and any combination thereof. Each of the aforementioned conditions can also have temporal limitations, for example, a requirement that the sensed energy increase to a qualifying maximum and then decrease to a threshold percentage of the maximum or by a threshold amount from the maximum within a predetermined amount of time.

If the signal passed to comparator 308 satisfies the threshold or condition, then comparator 308 outputs an indication of such to timing circuit 306. Timing circuit 306, like comparator 308, can be implemented with discrete or dedicated circuitry or by software executed by one or more processors. In addition to providing the control signal that strobes IR emitter 302 as discussed above, timing circuit 306 can include a clock that is used to time various actions taken by the electrical circuitry 310. Upon receiving the positive state indication (e.g., occurrence of a blink) from comparator 308, timing circuit 306 generates an outputs a signal to actuator driver 309, which is responsible for activating pump 124 and thereby causing the ejection of the therapeutic substance (shown here as a single drop 201) to the subject's eye. Actuator driver 309 can be configured for operation with the various types of pumps described herein. For example, in the case of a piezoelectric pump 124, actuator driver 309 can be a piezoamplifier. In some embodiments, the signal received at pump 124 is of a magnitude and duration that corresponds to the desired dosage (described in more detail below).

In certain embodiments, ejection of the therapeutic substance is desired to occur immediately after the detection of a blink. Thus, timing circuit 306 can be configured to output on the very next clock cycle after receipt of the positive state indication. In some cases, a small amount of delay is desirable to ensure that the blank has completed prior to ejection. There will be delays inherent in the operation of apparatus 101 itself which may be sufficient to generate the desired delay, but if additional delay is required then timing circuit 306 can be configured to output the actuator signal after a number of clock cycles have passed. This delay can be adjustable by a user to account for individuals that might have abnormally short or long blink times. Although not limited to such, this user adjustable delay can be up to 500 milliseconds.

Also depicted in FIG. 3 is a power supply 312, a user interface 314, communication circuitry 316 with one or more corresponding antennas 317, a display 318, a memory 322, and processing hardware 324. Power supply 312 can be one or more replaceable or rechargeable batteries that provide the operating power for apparatus 101. User interface 314 can include one or more user actuatable buttons, switches, dials, and the like that can accept user input for controlling the operation of apparatus 101. Examples can include a power on-off switch and one or more dials for adjusting the sensitivity of sensor 303 (such as to accommodate for variations in skin color), for setting the dosage to be delivered, or for adjusting the delay of ejection device 104. User interface 314 can also be implemented by way of display 318 in the form of a touchscreen. Display 318 can display the current settings of apparatus 101 to the user, operating instructions for the user, status messages for the user, or can provide feedback for any settings that have been adjusted by the user, to name a few.

Communication circuitry 316 includes the circuitry for communicating with external devices across either wired or wireless links. Communication circuitry 316 can include one or more ports for wired connection to a power source or an external computer (e.g., a USB or ethernet connection). Communication circuitry 316 can also provide wireless communication functionality according to any desired protocols, such as Bluetooth, Bluetooth Low Energy (BTLE), near field communication (NFC), Wi-Fi, and others. In certain embodiments, apparatus 101 is configured to interface with software running on a website, personal computer, PDA, or mobile communication device (e.g., a smartphone or wearable device like GOOGLE GLASS) and communication circuitry 316 provides this capability.

Processing hardware 324 can include one or more processors, microprocessors, controllers, microcontrollers, field programmable arrays (FPGAs), and the like, each of which can be a discrete chip or distributed amongst (and a portion of) a number of different chips. As noted above, comparator 308 and timing circuit 306, although shown separately, can be functionally implemented by processing hardware 324 and other components such as analog-to-digital and digital-to-analog converters (ADCs, DACs) and clocking circuitry (crystal oscillator, VCO, PLL, etc.).

Memory 322 can be shared by one or more of the various functional units present within apparatus 101, or can be distributed amongst two or more of them (e.g., as separate memories present within different chips). Memory 322 can also be a separate chip of its own. Memory 322 is non-transitory, and can be volatile (e.g., RAM, etc.) and/or non-volatile memory (e.g., ROM, flash memory, F-RAM, etc.).

Now referring back to FIG. 1, adapter unit 108 is configured to couple with a container for the therapeutic substance in a releasable fashion such as if adapter unit 108 is reusable, or in a non-releasable fashion such as if adapter unit 108 is for single-use. The container can be a standard dropper bottle in the form in which ocular drugs are commonly supplied. The subject couples the container to adapter unit 108, typically by screwing the container threads into corresponding threads of adapter 108, although other coupling interfaces can be used. Adapter unit 108 can include different thread geometries to couple with different types of containers. As mentioned, apparatus 101 may include one or more adapter units 108 depending on the number of therapeutic substances to be administered and whether administration is required for one or both eyes. In other embodiments adapter unit 108 can receive the therapeutic substance from a capsule or blister pack.

In still other embodiments adapter unit 108 can be omitted and therapeutic substances can be manually or mechanically supplied to one or more internal reservoirs 320, such as with a syringe. Numerous therapeutic substances can be supplied to apparatus 101 and mixed therein (such as within internal reservoir 320 of FIG. 3) to administer a combined solution to the eye. Also, ejection device 104 can include multiple nozzles 122 each supplied with a different therapeutic substance to simultaneously or sequentially deliver multiple doses to the eye.

Once the source of the therapeutic substance has been connected, apparatus 101 can be primed or filled with the fluid. Priming can be achieved by setting apparatus 101 in a priming mode where ejection device 104 draws the fluid into internal reservoir 320. Internal reservoir 320 can be a chamber connected with pump 124 or can be realized by the volume of fluid held within the internal fluidic channels of apparatus 101. The subject may squeeze the external container creating the pressure needed to fill apparatus 101 with fluid. In other embodiments, a prefilled priming reservoir may be used.

As mentioned, apparatus 101 may be used as part of a larger system 100 that includes other devices such as a smartphone or personal computer. A software application can be downloaded or installed, or a website can be utilized, that provides assistance to the user in performing dosage administration at the proper times and with the proper amounts. For example, a notification routine can be initiated that provides notification to the subject to take certain actions, such as to place apparatus 101 on the subject's face and begin a dosage administration procedure. The notification can be sent to the subject's phone in the form of a text message or email, or can be generated in visual, audible, and/or tactile (e.g., vibration) format on the subject's phone, tablet, or computer (typically not tactile).

Apparatus 101 can then be placed on the subject's face and, when the subject is ready for administration of the dosage, transitioned from the priming mode to a delivery mode. Placement on the face automatically orients sensor unit 106 so that emitter 302 and sensor 303 are focused on the eye of the subject enabling the opening and closure of the eye to be detected. Ejection device 104 is also automatically oriented such that nozzle 122 is aimed at the desired portion of the subject's eye, which may be either corner of the eye, the center of the eye, a center region of the eye, or a tear duct. If a correction of the alignment of sensor unit 106 or ejection device 104 is needed, the subject can manually adjust either unit 106 or device 104 as needed and then lock into place.

Apparatus 101 monitors for the occurrence of a blink. Once detected, after a small delay ejection device 104 is triggered causing the therapeutic substance to be propelled towards the eye. The therapeutic substance is propelled with sufficient velocity to overcome the force of gravity if the subject is in a sitting or standing position or looking downwards. Upon transitioning apparatus 101 to the delivery mode, a minor delay of a few seconds may be built into apparatus 101 to give the subject time to ready him or herself for administration of the dosage, which can include blinking the eye one or more times to adequately moisten it. An audible or other notification may be output by apparatus 101 once apparatus begins monitoring.

To facilitate detection of the blink, in some embodiments it can be desirable to include an optical light (e.g., an LED) or camera flash that, when set off, induces a blink at a point in time when apparatus 101 can expect it and then more easily sense it. The optical light can be part of apparatus 101 or, when used in conjunction with a smartphone, the smartphone's camera flash can be triggered by user or system 100 itself.

Details of the delivery such as dosage, dose frequency, response, and times may be logged and stored on apparatus 101 or broadcast wirelessly to one of the aforementioned external devices, at which point those details can be uploaded to a server and communicated to a medical professional. Responses to the delivery may be logged in a form of photograph through an optical imager attached to frame 102.

Molecular tags such as fluorophores may be utilized in the therapeutic substance linked to physiological markers to provide information regarding the dose, pharmacokinetics, or pharmacodynamics. Any of multiple wavelengths may be used for detection and excitation of different biomarkers. Dosage can then be adjusted, e.g., in real time, to match a particular pharmacodynamic or physiological response. This data may be used to indicate when to administer the medicament. In certain embodiments, apparatus 101 can include an opthalmoscope.

As mentioned above, apparatus 101 can deliver the entire desired dosage with a single drop (or continuous fluid body). The use of a single drop having a known volume provides greater confidence that the entire volume with be transferred to the subject's eye, as compared to mists, fogs, and even focused, jet-like sprays that break the dosage down into many small particulates to transfer to the eye.

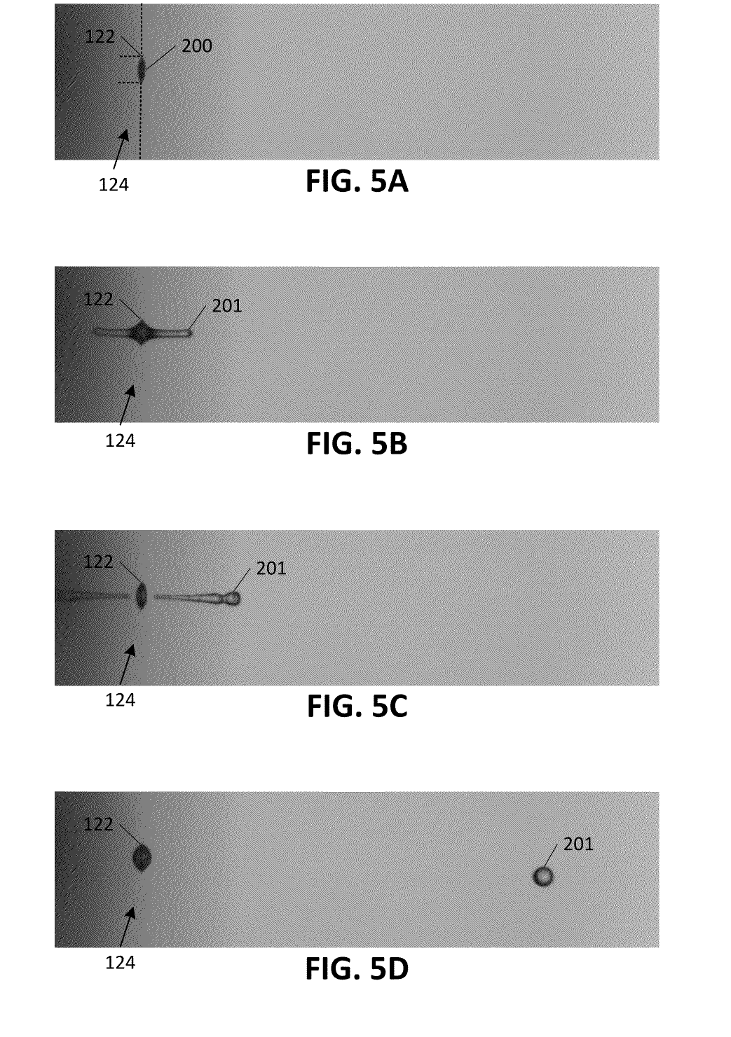

FIGS. 5A-D are close-up photographs of pump 124 with nozzle 122 during formation and ejection of single drop 201. FIG. 5A depicts the leading edge of the therapeutic substance 200 prior to actuation of pump 124. (The leading edge is reflected on the surface of the pump and this gives the substance a cat-eye shape.) Black lines are overlaid on the photograph to aid in illustrating the leading edge of the pump surface and the interior edges of nozzle 122 (which are obscured).

The embodiment pictured here has a pump wall 132 configured as a membrane (not shown). FIG. 5B depicts pump 124 as the internal pump membrane is being actuated and moving in a left to right direction. This movement causes a generally columnal, elongate drop of fluid 201 (resembling a jet or stream) to be forced from nozzle 122 in the same direction. Drop 201 has a diameter approximate to the diameter of nozzle 122. The length of drop 201 will vary, but at this point in time is about 6-7 times the diameter of drop 201.

FIG. 5C depicts pump 124 after movement of the pump membrane (not shown) has ceased. Cessation of membrane movement is preferably performed quickly such that the pressure within chamber 130 (not shown) is rapidly reduced to its original level (or a negative level that would refill the pump). The rapid ejecting movement of the membrane followed by the rapid (or substantially instantaneous) cessation of movement of the membrane allows the amount of fluid ejected through nozzle 122, and therefore the dosage, to be precisely controlled.

As seen in FIG. 5C, drop 201 assumes a comet-like shape owing to air resistance. The leading surface of drop 201 bulges to a width greater than that of nozzle 122 and the rear portion of drop 201 takes on a tapered shape. Undulations in the surface of drop 201 may occur, such as the formation of the narrow neck shown behind the leading bulge. For larger doses, this may result in the splitting of drop 201 into two or more smaller drops. However, the drop does not split into a substantial number of particulates (e.g., more than 100) as in some conventional devices.

FIG. 5D depicts drop 201 after progressing further from nozzle 122. Here, drop 201 has transitioned from the comet-like shape of FIG. 5C to a substantially spherical shape due to the surface tension of the constituent substance. Drop 201 continues in this manner until colliding with the subject's eye. The leading edge of the next drop 201 is shown in nozzle 122. The pressure within pump 124 is held constant to prevent the therapeutic substance from leaking or “dribbling” out.

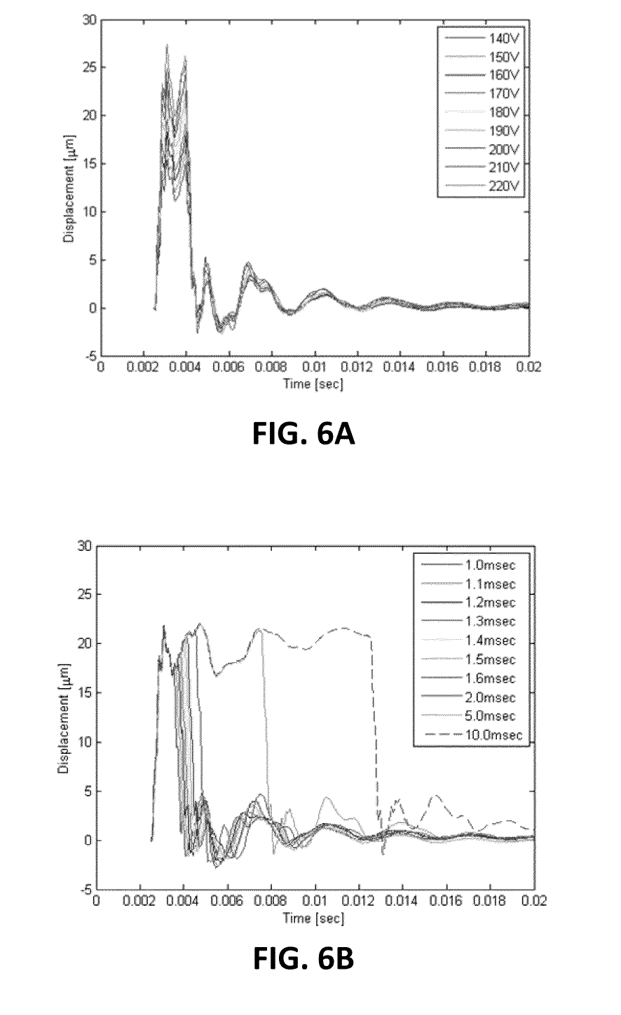

FIG. 6A is a graph depicting the change in displacement of an example pump membrane in response to membrane actuator signals having approximately the same duration but different magnitudes. FIG. 6B is a graph depicting the change in displacement of an example pump membrane in response to actuation signals of approximately the same magnitude but having different durations. The change in displacement of the pump membrane and the time in which the membrane remains displaced correlate to the volume of the dosage administered, e.g., the volume of the single drop. Thus the subject can enter in a dosage amount into user interface 314, which is taken by apparatus 101 and translated to a membrane actuator signal of the appropriate voltage and duration to provide that specified dosage amount. The displaceable pump wall 132 or 212 preferably displaces in rapid fashion to increase the controllability of ejection. In these examples, the membrane wall displaces the maximum amount in approximately one-tenth of a micro-second.

Certain subject matter described herein was arrived at through a joint research agreement between the Regents of the University of California (San Francisco Campus) and the California Institute of Technology.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Express recitation of every possible combination and substitution is overly burdensome, especially given that the permissibility of each and every such combination and substitution will be readily recognized by those of ordinary skill in the art upon reading this description.

Where a range of values is provided, it is noted that each intervening value, to the tenth of the unit of the lower limit unless the context clearly dictates otherwise, between the upper and lower limit of that range and any other stated or intervening value in that stated range, is encompassed within the disclosure and can be claimed as an sole value or as a smaller range. Where the stated range includes one or both of the limits, ranges excluding either or both of those included limits are also included in the disclosure.

Where a discrete value or range of values is provided, it is noted that that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term.

However, in no way should a claim be limited to a particular value or range of values absent explicit recitation of that value or range of values in the claims. Values and ranges of values are provided herein merely as examples.

In some instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

As used herein and in the appended claims, the singular forms “a”, “an”, and “the” include plural referents unless the context clearly dictates otherwise.

The scope of the claims originally filed herewith is not intended to define the limits of the subject matter that may be claimed on the basis of this description. Broader and/or altogether different subject matter may, in fact, be claimed in the future without departing from the scope of the present description.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. An apparatus for the delivery of a therapeutic substance to an eye of a human subject, comprising:
an ejection device adapted to dispense the therapeutic substance to the eye of the subject;
a sensor coupled with the ejection device; and
control circuitry communicatively coupled with the sensor and configured to determine a condition indicative of a current or future open state of the eye based upon information received from the sensor, the control circuitry further configured to actuate ejection of the therapeutic substance by the ejection device after the condition is detected.

2. The apparatus of claim 1, further comprising a wearable frame, wherein the ejection device, sensor, and control circuitry are coupled with the wearable frame.

3-5. (canceled)

6. The apparatus of claim 1, further comprising an emitter adapted to emit a signal towards the eye of the subject, wherein the sensor is adapted to sense the signal after reflection from the subject.

7. The apparatus of claim 6, wherein the signal is an infrared signal.

8. The apparatus of claim 7, wherein the control circuitry is configured to cause the emitter to emit the infrared signal in pulses at a frequency in the range of about 500 hertz to about 60 kilohertz.

9. The apparatus of claim 8, wherein the control circuitry is configured to filter and amplify a signal output from the sensor to detect the presence of the pulsed infrared signal.

10. The apparatus of claim 9, wherein the condition is an increase in the amount of the infrared signal sensed by the sensor followed by a decrease in the amount of the infrared signal sensed by the sensor.

11. The apparatus of claim 9, wherein the condition is an increase in the amount of the infrared signal sensed by the sensor followed by a decrease in the amount of the infrared signal sensed by the sensor to a threshold value.

12. The apparatus of claim 1, wherein the control circuitry is configured to actuate the ejection device after determining the occurrence of the condition.

13. The apparatus of claim 12, wherein the control circuitry is configured to actuate the ejection device up to 500 milliseconds after determining the occurrence of the blink.

14. The apparatus of claim 12, wherein the control circuitry is configured to actuate the ejection device up to 300 milliseconds after determining the occurrence of the blink.

15. The apparatus of claim 12, adapted to allow the user to adjust a delay between determining the occurrence of the blink and actuating the ejection device.

16. The apparatus of claim 11, wherein the control circuitry is configured to actuate the ejection device after determining the occurrence of movement of an eyelid of the subject from an open position to a closed position.

17. The apparatus of claim 1, wherein the control circuitry is configured to actuate the ejection device after determining the occurrence of movement of an eyelid of the subject from a closed position to an open position.

18. The apparatus of claim 1, wherein the control circuitry is configured to actuate the ejection device after determining the occurrence of movement of an eyelid of the subject from an open position to a closed position and then back to an open position.

19. The apparatus of claim 1, wherein the ejection device comprises only a single aperture for ejecting the therapeutic substance.

20. The apparatus of claim 19, wherein the ejection device is configured to eject a single drop of the therapeutic substance through the single aperture at a velocity sufficient to transfer the single drop to the eye.

21. (canceled)

22. The apparatus of claim 20, wherein the ejection device is controllable to adjust the volume of therapeutic substance contained in the single drop.

23-24. (canceled)

25. The apparatus of claim 1, wherein the apparatus is not adapted to eject the therapeutic substance to the eye in the form of a mist.

26. The apparatus of claim 1, wherein the apparatus is not adapted to eject the therapeutic substance to the eye in the form of a stream comprising a substantial number of particulates.

27-46. (canceled)
